(19) United States
(12) Patent Application Publication
(10) Pub. No.: US 2026/0020897 A1
(43) Pub. Date: Jan. 22, 2026

(54) TREATMENT TOOL AND MANUFACTURING METHOD

(71) Applicant: OLYMPUS MEDICAL SYSTEMS CORP., Tokyo (JP)

(72) Inventor: Tsunetaka Akagane, Tokyo (JP)

(21) Appl. No.: 19/008,176

(22) Filed: Jan. 2, 2025

Related U.S. Application Data
(63) Continuation of application No. PCT/JP2024/025705, filed on Jul. 17, 2024.

(51) Int. Cl. A61B 18/14; A61B 17/00; A61B 18/00

(52) U.S. Cl. CPC A61B 18/1445; A61B 2017/00526; A61B 2018/0013; A61B 2018/00589; A61B 2018/00601; A61B 2018/00607; A61B 2018/00994

(57) ABSTRACT

A treatment tool includes: a treatment surface for treating of a treatment target; and a plurality of recessed portions that are provided in at least a part of the treatment surface and in which first resins are disposed, the treatment surface including a surface region not covered with the first resins, the surface region functioning as a first electrode supplying a high-frequency current to the treatment target to treat the treatment target.

### CROSS REFERENCES TO RELATED APPLICATIONS

This application is a continuation of International Application No. PCT/JP2024/025705, filed on Jul. 17, 2024, the entire contents of which are incorporated herein by reference.

### BACKGROUND

### 1. Technical Field

The present disclosure relates to a treatment tool and a manufacturing method.

### 2. Description of the Related Art

In the related art, there has been known a treatment tool configured to treat a treatment target by applying treatment energy to a portion which is a target to be treated in a living tissue (hereinafter, referred to as a treatment target) (for example, refer to JP 2011-505198 A).

In the treatment tool described in JP 2011-505198 A, ultrasonic energy (ultrasonic vibration) is adopted as treatment energy.

### SUMMARY

In some embodiments, a treatment tool includes: a treatment surface for treating of a treatment target; and a plurality of recessed portions that are provided in at least a part of the treatment surface and in which first resins are disposed, the treatment surface including a surface region not covered with the first resins, the surface region functioning as a first electrode supplying a high-frequency current to the treatment target to treat the treatment target.

In some embodiments, provided is a method of manufacturing a treatment tool including a treatment surface for treatment of a treatment target. The method includes: providing a plurality of recessed portions in the treatment surface; applying first resins to the treatment surface; and removing, among the first resins applied to the treatment surface, resins other than the first resins disposed in the plurality of recessed portions.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a treatment system according to an embodiment;

FIG. 2 is a diagram for explaining a configuration of a distal end portion in a treatment tool;

FIG. 3 is a diagram for explaining a configuration of a jaw and a vibration transmission portion;

FIG. 4 is a diagram for explaining a structure of an outer surface of the vibration transmission portion;

FIG. 5 is a diagram for explaining the structure of the outer surface of the vibration transmission portion;

FIGS. 6A, 6B and 6C are diagrams for explaining a method of forming a heat insulation reinforcement portion;

FIG. 7 is a diagram illustrating a first modification of the embodiment;

FIG. 8 is a diagram illustrating a second modification of the embodiment;

FIG. 9 is a diagram illustrating a third modification of the embodiment;

FIG. 10 is a diagram illustrating a fourth modification of the embodiment;

FIG. 11 is a diagram illustrating the fourth modification of the embodiment; and

FIG. 12 is a diagram illustrating the fourth modification of the embodiment.

### DETAILED DESCRIPTION

Hereinafter, embodiments will be described with reference to the drawings. It is noted that the disclosure is not limited by the embodiments described below. Furthermore, in the description of the drawings, the same portions will be denoted by the same reference numerals.

### Schematic Configuration of Treatment System

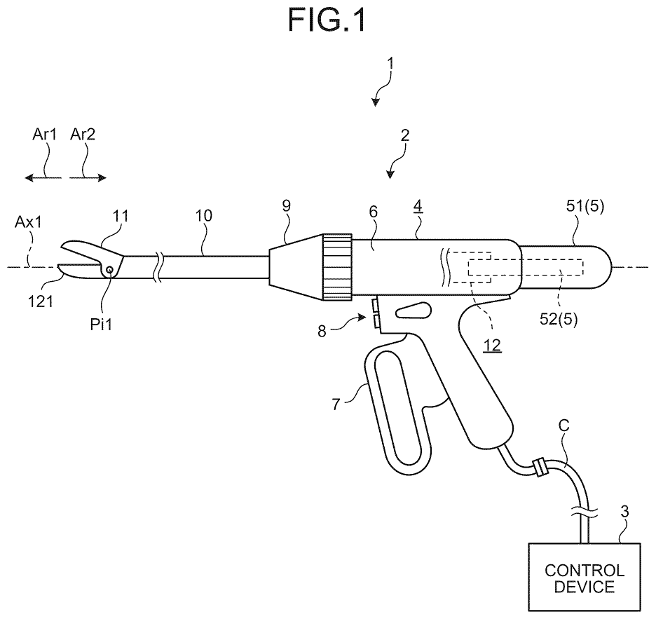

FIG. 1 is a diagram illustrating a treatment system 1 according to an embodiment.

The treatment system 1 applies treatment energy to a portion to be treated (hereinafter, referred to as a treatment target) in a living tissue, thereby treating the treatment target. It is noted that the treatment energy in the present embodiment is ultrasonic energy and high-frequency energy. In addition, the treatment that can be performed by the treatment system 1 according to the present embodiment is treatment such as coagulation (sealing) of the treatment target and incision of the treatment target. Further, coagulation and incision may be performed simultaneously. It is noted that the treatment energy applied to the treatment target is not limited to both the ultrasonic energy and the high-frequency energy, and may be only the high-frequency energy. As illustrated in FIG. 1, the treatment system 1 includes a treatment tool 2 and a control device 3.

### Configuration of Treatment Tool

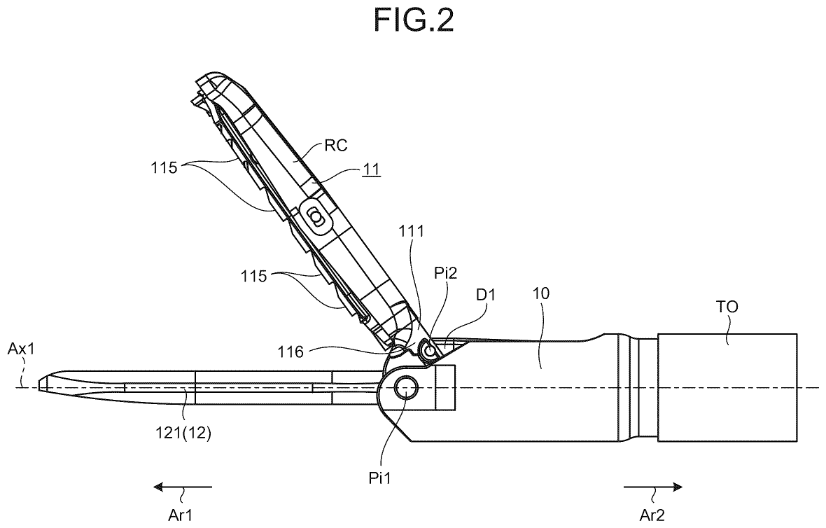

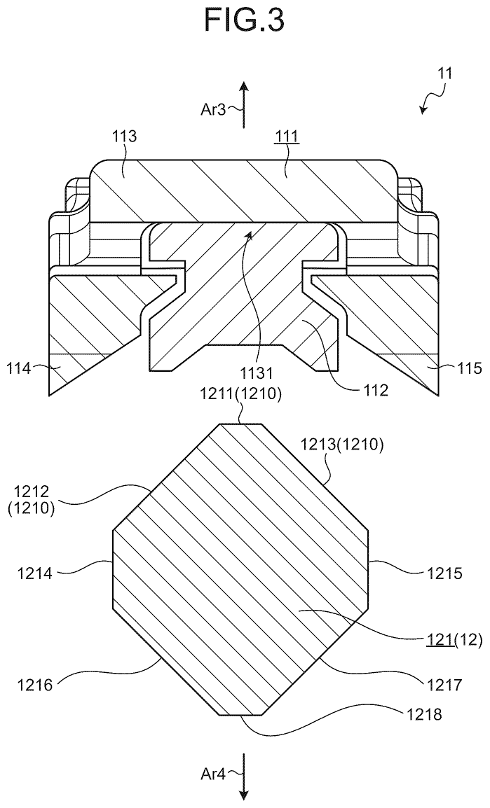

It is noted that, hereinafter, one side along a central axis Ax1 of a sheath 10 is referred to as a distal end side Ar1, and the other side is referred to as a proximal end side Ar2 (FIG. 1). Further, a description will be given below. In addition, a “width direction” is a direction orthogonal to the central axis Ax1 and the opening-and-closing direction of a jaw 11 with respect to a treatment portion 121, and means a direction orthogonal to the paper surfaces of FIGS. 1 and 2, and the left-and-right direction of FIG. 3.

FIG. 2 is a diagram illustrating a configuration of a distal end portion of the treatment tool 2. Specifically, FIG. 2 is a diagram illustrating the distal end portion of the treatment tool 2 when viewed in the width direction.

The treatment tool 2 is a treatment tool configured to treat a treatment target by applying ultrasonic energy and high-frequency energy to the treatment target. As illustrated in FIG. 1, the treatment tool 2 includes a handpiece 4 and an ultrasonic transducer 5.

As illustrated in FIGS. 1 to 3, the handpiece 4 includes a holding case 6 (FIG. 1), an operation handle 7 (FIG. 1), a switch 8 (FIG. 1), a rotary knob 9 (FIG. 1), the sheath 10, the jaw 11, and a vibration transmission portion 12.

The holding case 6 supports the entire treatment tool 2.

The operation handle 7 is movably attached to the holding case 6 and receives opening and closing operations by an operator such as a technician.

The switch 8 is provided in a state of being exposed to the outside of the holding case 6, and receives a treatment operation by an operator such as a technician.

The rotary knob 9 has a substantially cylindrical shape coaxial with the central axis Ax1, and is provided on the distal end side Ar1 of the holding case 6. Then, the rotary knob 9 receives a rotation operation by an operator such as a technician. By the rotation operation, the rotary knob 9 rotates around the central axis Ax1 with respect to the holding case 6. The rotation of the rotary knob 9 rotates the sheath 10, the jaw 11, and the vibration transmission portion 12 around the central axis Ax1.

The sheath 10 is a cylindrical pipe made of an electrically conductive material such as metal.

In the sheath 10, a columnar first pin Pi1 (FIGS. 1 and 2) extending in the width direction is fixed to an end portion on the distal end side Ar1.

The outer peripheral surface of the sheath 10 is covered with an electrically insulating outer tube TO (FIG. 2). Further, the inner peripheral surface of the sheath 10 is covered with an electrically insulating inner tube TI (not illustrated).

FIG. 3 is a diagram for explaining a configuration of the jaw 11 and the vibration transmission portion 12. Specifically, FIG. 3 is a cross-sectional view of the jaw 11 and the vibration transmission portion 12 taken along a plane orthogonal to the central axis Ax1. It is noted that, in FIG. 3, illustration of a cover RC is omitted.

Hereinafter, in the description of the configuration of the jaw 11 and the vibration transmission portion 12, in the opening-and-closing direction of the jaw 11 with respect to the treatment portion 121, the opening direction (upper side in FIG. 3) of the jaw 11 is referred to as an open side Ar3, and the closing direction (lower side in FIG. 3) of the jaw 11 is referred to as a closed side Ar4.

The jaw 11 is pivotally supported by a first pin Pi1 with respect to the end portion of the distal end side Ar1 of the sheath 10, and is configured to be rotatable about the central axis (axis in the direction orthogonal to the paper surface of FIGS. 1 and 2) of the first pin Pi1. Then, the jaw 11 rotates about the central axis of the first pin Pi1 to be opened and closed with respect to the treatment portion 121 provided at the end portion on the distal end side Ar1 of the vibration transmission portion 12. Here, when the jaw 11 is closed with respect to the treatment portion 121, the treatment target is gripped between the jaw 11 and the treatment portion 121.

As illustrated in FIG. 3, the jaw 11 includes a jaw body 111 and a pad 112.

The jaw body 111 is made of an electrically conductive material. As illustrated in FIGS. 2 and 3, the jaw body 111 is a member in which a base body 113 (FIG. 3), a plurality of first tooth portions 114 (FIG. 3), a plurality of second tooth portions 115 (FIG. 3), and a bearing portion 116 (FIG. 2) are formed to be integrated with each other.

The base body 113 is formed of a substantially long plate body. In the present embodiment, the longitudinal direction of the base body 113 is a direction along a curve toward the left side as it goes toward the distal end side Ar1 when viewed from the proximal end side Ar2 in a state in which the jaw 11 is located on the upper side with respect to the treatment portion 121.

A cover RC (FIG. 2) made of an electrically insulating resin is integrally formed on the surface of the open side Ar3 of the base body 113 in a state of covering the surface of the open side Ar3. It is noted that, in the present embodiment, the cover RC is insert-molded with respect to the base body 113, but the disclosure is not limited thereto. For example, a configuration in which the cover RC is fixed to the base body 113 by a snap-fit or a metal pin may be adopted.

The plurality of first tooth portions 114 protrude from one side in the width direction of the surface of the closed side Ar4 of the base body 113 toward the closed side Ar4, and are arranged side by side in the longitudinal direction of the base body 113.

The plurality of second tooth portions 115 protrude from the other side in the width direction of the surface of the closed side Ar4 of the base body 113 toward the closed side Ar4, and are arranged side by side in the longitudinal direction of the base body 113.

Here, in the surface of the base body 113 on the closed side Ar4, at the central portion located between the plurality of first tooth portions 114 and the plurality of second tooth portions 115 in the width direction, as illustrated in FIG. 3, a recessed groove 1131 recessed toward the open side Ar3 and formed to extend in the longitudinal direction of the base body 113 is provided.

The bearing portion 116 is a portion that is provided at the proximal end of the base body 113 and is pivotally supported with respect to the sheath 10 by the first pin Pi1.

A columnar second pin Pi2 (FIG. 2) extending in the width direction is fixed to the bearing portion 116 by welding. The second pin Pi2 is connected to an opening and closing mechanism D1 (FIG. 2) inserted into the sheath 10. Then, the jaw 11 rotates about the central axis of the first pin Pi1 in conjunction with movement of the opening and closing mechanism D1 to the distal end side Ar1 or the proximal end side Ar2 according to the opening and closing operations to the operation handle 7 by an operator such as a technician, and is opened and closed with respect to the treatment portion 121.

The pad 112 is made of a resin material having electrical insulation and biocompatibility, for example, polytetrafluoroethylene (PTFE), and has a substantially rectangular parallelepiped shape extending in the longitudinal direction of the base body 113. As illustrated in FIG. 3, the pad 112 is fixed to the recessed groove 1131 of the base body 113.

When the jaw 11 is brought close to the treatment portion 121, the pad 112 abuts on the treatment portion 121.

The vibration transmission portion 12 has an elongated shape and is made of an electrically conductive material such as a titanium alloy. In addition, as illustrated in FIG. 2, the vibration transmission portion 12 is inserted into the sheath 10 in a state in which the treatment portion 121 protrudes to the outside. At this time, as illustrated in FIG. 1, the end portion of the vibration transmission portion 12 on the proximal end side Ar2 is mechanically connected to an ultrasonic transducer unit 52 constituting the ultrasonic transducer 5. Then, the vibration transmission portion 12 transmits the ultrasonic vibration generated by the ultrasonic transducer 5 from the end portion on the proximal end side Ar2 to the treatment portion 121. In the present embodiment, the ultrasonic vibration is longitudinal vibration that vibrates in a direction along the central axis Ax1.

In the present embodiment, similarly to the jaw 11, the treatment portion 121 extends along a curve toward the left side as it goes toward the distal end side Ar1 as viewed from the proximal end side Ar2 in a state in which the jaw 11 is located on the upper side. As illustrated in FIG. 3, the treatment portion 121 has a substantially octagonal cross section taken along a plane orthogonal to the central axis Ax1. It is noted that the octagonal shape, which is the cross-sectional shape of the treatment portion 121, is merely an example, and other shapes such as a circular shape may be used. Hereinafter, for convenience of description, the cross-sectional shape of the treatment portion 121 will be described as an octagonal shape.

Hereinafter, in the treatment portion 121, the flat surface located on the open side Ar3 is referred to as a first surface 1211. The first surface 1211 is a surface that abuts on the pad 112 when the jaw 11 is closed with respect to the treatment portion 121. In addition, surfaces adjacent to the first surface 1211 in the circumferential direction around the central axis of the treatment portion 121 are referred to as second and third surfaces 1212 and 1213, respectively. Further, surfaces adjacent to the second and third surfaces 1212 and 1213 in the circumferential direction around the central axis of the treatment portion 121 are referred to as fourth and fifth surfaces 1214 and 1215, respectively. In addition, surfaces adjacent to the fourth and fifth surfaces 1214 and 1215 in the circumferential direction around the central axis of the treatment portion 121 are referred to as sixth and seventh surfaces 1216 and 1217, respectively. Further, a surface located between the sixth and seventh surfaces 1216 and 1217 and positioned on the opposite side of the first surface 1211 is referred to as an eighth surface 1218.

The first to third surfaces 1211 to 1213 described above are surfaces for treating a treatment target and correspond to a treatment surface 1210 (FIG. 3). The first surface 1211 corresponds to an abutting surface.

It is noted that a detailed structure of the outer surface of the vibration transmission portion 12 including the treatment portion 121 will be described in “Structure of Outer Surface of Vibration Transmission portion” to be described later.

As illustrated in FIG. 1, the ultrasonic transducer 5 includes a TD (transducer) case 51 and the ultrasonic transducer unit 52.

The TD case 51 supports the ultrasonic transducer unit 52 and is detachably connected to the holding case 6.

The ultrasonic transducer unit 52 generates ultrasonic vibration under the control of the control device 3. In the present embodiment, the ultrasonic transducer unit 52 is a bolted Langevin type transducer (BLT).

### Configuration of Control Device

The control device 3 integrally controls the operations of the treatment tool 2 via an electric cable C (FIG. 1).

Specifically, the control device 3 detects a treatment operation on the switch 8 by an operator such as a technician via the electric cable C. Then, upon detecting the treatment operation, the control device 3 applies treatment energy to a treatment target gripped between the jaw 11 and the treatment portion 121 via the electric cable C. That is, the control device 3 treats the treatment target.

For example, when applying the ultrasonic energy to the treatment target, the control device 3 supplies drive power to the ultrasonic transducer unit 52 via the electric cable C. As a result, the ultrasonic transducer unit 52 generates longitudinal vibration (ultrasonic vibration) that vibrates in a direction along the central axis Ax1. In addition, the treatment portion 121 vibrates with a desired amplitude by the longitudinal vibration. Then, ultrasonic vibration is applied from the treatment portion 121 to the treatment target gripped between the jaw 11 (pad 112) and the treatment portion 121. In other words, the ultrasonic energy is applied from the treatment portion 121 to the treatment target.

In addition, for example, when applying high-frequency energy to the treatment target, the control device 3 supplies high-frequency power between the jaw body 111 and the vibration transmission portion 12 via the electric cable C. When the high-frequency power is supplied between the jaw body 111 and the vibration transmission portion 12, the high-frequency current is supplied to the treatment target located between the jaw body 111 and the treatment portion 121. In other words, the high-frequency energy is applied to the treatment target.

### Structure of Outer Surface of Vibration

### Transmission Portion

Next, the structure of the outer surface of the vibration transmission portion 12 including the treatment portion 121 will be described.

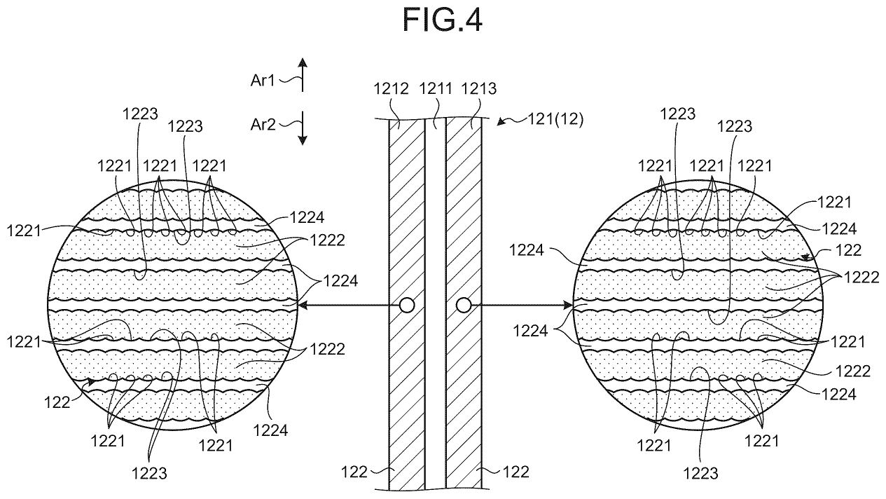

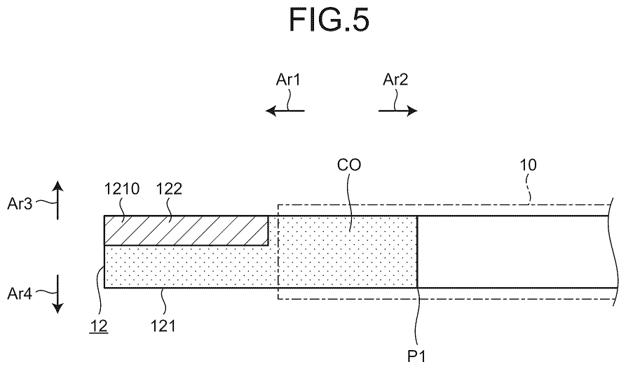

FIGS. 4 and 5 are diagrams for explaining the structure of the outer surface of the vibration transmission portion 12. Specifically, FIG. 4 is a diagram of the vibration transmission portion 12 viewed from the first surface 1211 side along the normal direction of the first surface 1211. It is noted that, in FIG. 4, a portion in which a heat insulation reinforcement portion 122 to be described later is provided is hatched. FIG. 5 is a diagram of the distal end portion of the vibration transmission portion 12 as viewed in the width direction. It is noted that, in FIG. 5, a portion in which the heat insulation reinforcement portion 122 is provided is hatched. In FIG. 5, a dot is added to a portion in which a coating layer CO to be described later is provided. Furthermore, in FIG. 5, the sheath 10 is illustrated by a one-dot chain line.

The heat insulation reinforcement portion 122 is a portion in which a heat insulation effect on the treatment surface 1210 is enhanced. In the present embodiment, as illustrated in FIG. 4, the heat insulation reinforcement portion 122 is provided in the second and third surfaces 1212 and 1213 of the treatment surface 1210 excluding the first surface 1211.

As illustrated in FIG. 4, the heat insulation reinforcement portion 122 includes a plurality of recessed portions 1221, and first resins 1222 respectively disposed in the plurality of recessed portions 1221. It is noted that, in FIG. 4, a dot is added to a portion in which the first resins 1222 are disposed.

In the present embodiment, the plurality of recessed portion 1221 are respectively formed in the second and third surfaces 1212 and 1213 by laser processing using a short pulse laser, and each of the recessed portions has a circular shape. In the example of FIG. 4, a plurality of rows (hereinafter, referred to as a groove portion 1223) of the plurality of recessed portions 1221 arranged in a state in which the plurality of recessed portions 1221 overlap each other in a direction orthogonal to the longitudinal direction of the vibration transmission portion 12 (in a state of communicating with each other) are arranged in parallel with each other in the longitudinal direction.

It is noted that, hereinafter, in the second and third surfaces 1212 and 1213, a portion other than the portion in which the groove portion 1223 is formed is referred to as a surface 1224 (FIG. 4).

Each of the first resins 1222 is an electrically insulating material, and is made of a fluororesin such as polyether ether ketone (PEEK), polyimide (PI), perfluoroalkoxy alkane (PFA), or polytetrafluoroethylene (PTFE). The first resins 1222 are respectively disposed inside the groove portions 1223 in a state of being substantially flush with the surface 1224.

A method of forming the heat insulation reinforcement portion 122 described above will be described in “Method of Forming Heat Insulation Reinforcement Portion” to be described later.

Then, in the present embodiment, the surfaces 1211 and 1224 of the treatment surface 1210, which are not covered with the first resins 1222, function as first electrodes that treat the treatment target by supplying the high-frequency current to the treatment target without limiting the path of the high-frequency current to the treatment target by the first resins 1222. In addition, the jaw body 111 functions as a second electrode that supplies a high-frequency current to the treatment target between the first electrode and the jaw body.

A coating layer CO is a layer coated with a second resin. Examples of the coating layer CO (second resin) include fluororesins such as PEEK, PI, PFA, and PTFE, which are materials having electrical insulation properties. In the present embodiment, as illustrated in FIG. 5, the coating layer CO is provided on the outer surface (including the fourth to eighth surfaces 1214 to 1218) excluding the treatment surface 1210 and being located on the distal end side Ar1 from a node position P1 on the most distal end side Ar1 among the node positions of the longitudinal vibration in the vibration transmission portion 12.

### Method of Forming Heat Insulation Reinforcement Portion

Next, a method of forming the heat insulation reinforcement portion 122 will be described. It is noted that the formation method corresponds to the manufacturing method of the treatment tool 2.

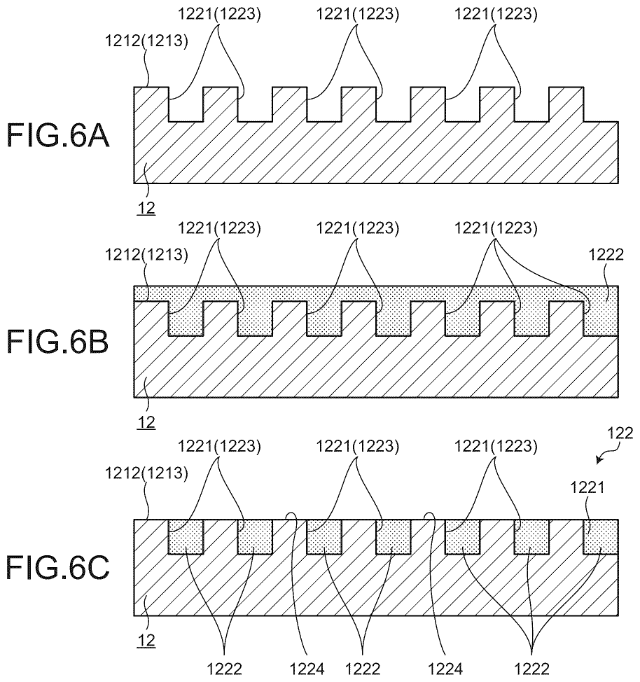

FIGS. 6A, 6B and 6C are diagrams illustrating a method of forming the heat insulation reinforcement portion 122. Specifically, FIGS. 6A, 6B and 6C are cross-sectional views of the vibration transmission portion 12 taken along a plane orthogonal to the extending direction of the groove portion 1223.

First, as illustrated in FIG. 6A, an operator forms a plurality of recessed portions 1221 (groove portions 1223) in the second and third surfaces 1212 and 1213 by, for example, laser processing (hereinafter, referred to as a first step).

Next, as illustrated in FIG. 6B, the operator applies the uncured first resins 1222 to the second and third surfaces 1212 and 1213 (hereinafter, referred to as a second step).

Next, as illustrated in FIG. 6C, the operator removes, among the first resins 1222 applied to the second and third surfaces 1212 and 1213, the first resins 1222 other than the first resins respectively disposed inside the plurality of recessed portions 1221 (groove portions 1223) (hereinafter, referred to as a third step).

In the third step, for example, as described below, the first resins 1222 other than the first resins respectively disposed inside the plurality of recessed portions 1221 are removed.

For example, when the first resins 1222 are in an uncured state, the operator removes the first resins 1222 other than the first resins respectively disposed inside the plurality of recessed portions 1221 by tracing the second and third surfaces 1212 and 1213 with a spatula or the like.

For example, after the first resins 1222 are cured, the operator removes the first resins 1222 other than the first resins respectively disposed inside the plurality of recessed portions 1221 by polishing such as buffing.

It is noted that, for example, when the first resins 1222 and the coating layer CO are made of the same material, the heat insulation reinforcement portion 122 and the coating layer CO can be formed as follows.

For example, after the first step, in the second step, in a state in which the first surface 1211 is masked, the uncured first resins 1222 are applied to the entire outer surface of the vibration transmission portion 12 on the distal end side Ar1 from the node position P1. Then, in the third step, among the first resins 1222 present on the second and third surfaces 1212 and 1213, the first resins 1222 other than the first resins respectively disposed inside the plurality of recessed portions 1221 (groove portions 1223) are removed.

In addition, for example, after the first step, in the second step, the uncured first resins 1222 are applied to the entire outer surface of the vibration transmission portion 12 on the distal end side Ar1 from the node position P1. Then, in the third step, the first resins 1222 present on the first surface 1211 are removed, and, among the first resins 1222 present on the second and third surfaces 1212 and 1213, the first resins 1222 other than the first resins respectively disposed inside the plurality of recessed portions 1221 (groove portions 1223) are removed.

According to the present embodiment described above, the following effects are obtained.

In the treatment tool 2 according to the present embodiment, at least a part of the treatment surface 1210 is provided with a plurality of recessed portions 1221 in which the first resins 1222 are respectively disposed. The surfaces 1211 and 1224 of the treatment surface 1210, which are not covered with the first resins 1222, function as first electrodes that treat the treatment target by supplying the high-frequency current to the treatment target without limiting the path of the high-frequency current to the treatment target by the first resins 1222.

Therefore, according to the treatment tool 2 of the present embodiment, while residual heat of the treatment surface 1210 can be reduced by the heat insulation reinforcement portion 122 provided on a part of the treatment surface 1210, a high-frequency current can be supplied to the treatment target.

In particular, the first resins 1222 are respectively disposed inside the recessed portion 1221. Therefore, as compared with a configuration in which the first resins 1222 are simply provided on the treatment surface 1210, it is possible to realize a structure in which the first resins 1222 are hardly removed even by friction due to ultrasonic vibration. That is, the durability of the heat insulation reinforcement portion 122 can be improved.

In the treatment tool 2 according to the present embodiment, the coating layer CO is provided on the outer surface of the vibration transmission portion 12 on the distal end side Ar1 from the most distal node position P1 except for the treatment surface 1210. Therefore, it is possible to further reduce the influence of thermal invasion on an unintended region in a living tissue.

Furthermore, in the treatment tool 2 according to the present embodiment, when the jaw 11 is closed with respect to the treatment surface 1210, the heat insulation reinforcement portion 122 is not provided in the first surface 1211 in contact with the jaw 11. In other words, the heat insulation reinforcement portion 122 is not provided in the first surface 1211 where there is a risk that the first resins 1222 may be removed due to friction caused by ultrasonic vibration on the treatment surface 1210. Therefore, the heat insulation reinforcement portion 122 can be provided only at an appropriate position.

### Other Embodiments

Although the embodiment has been described so far, the disclosure should not be limited only by the above-described embodiment. In the above-described embodiment, the groove portions 1223 are formed in a state in which a plurality of recessed portions 1221 overlap each other, but the disclosure is not limited thereto, and the plurality of recessed portions 1221 may be provided in a state of not overlapping each other.

In the above-described embodiment, the first resins 1222 and the coating layer CO have a function as a heat insulating coating, but the disclosure is not limited thereto, and the first resins 1222 and the coating layer CO may have a function as an anti-sticking coating.

In the above-described embodiment, the opening and closing mechanism D1 is provided inside the sheath 10, but the disclosure is not limited thereto. For example, the sheath 10 itself may move to the distal end side Ar1 or the proximal end side Ar2 to open and close the jaw 11 with respect to the treatment portion 121. In addition, the opening and closing of the jaw 11 may be configured to be closed with respect to the treatment portion 121 when the opening and closing mechanism D1 and the sheath 10 move to the distal end side Ar1, or may be configured to be closed with respect to the treatment portion 121 when the opening and closing mechanism D1 and the sheath 10 move to the proximal end side Ar2.

In the above-described embodiment, the ultrasonic transducer 5 is configured to be detachable from the handpiece 4, but the disclosure is not limited thereto, and a configuration in which the ultrasonic transducer 5 is incorporated into the handpiece 4 may be adopted.

In the above-described embodiment, the number of switches 8 is not limited to two illustrated in FIG. 1, and may be one or three or more.

In the above-described embodiment, when high-frequency power is supplied between the jaw 11 and the vibration transmission portion 12, the jaw 11 side may be used as a reference voltage, and conversely, the vibration transmission portion 12 side may be used as a reference voltage.

In the above-described embodiment, first to fourth modifications described below may be adopted.

### First Modification

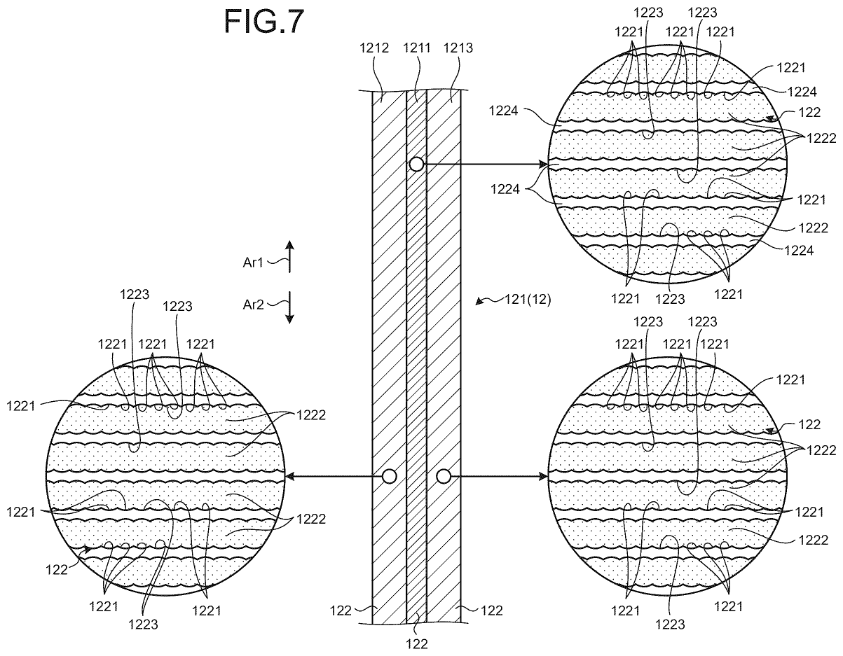

FIG. 7 is a diagram illustrating a first modification of the embodiment. Specifically, FIG. 7 is a diagram corresponding to FIG. 4. It is noted that, in FIG. 7, a portion in which the heat insulation reinforcement portion 122 is provided is hatched. In addition, in FIG. 7, a dot is added to a portion in which the first resin 1222 is disposed.

In the above-described embodiment, the heat insulation reinforcement portion 122 is provided in the second and third surfaces 1212 and 1213 of the treatment surface 1210, but the disclosure is not limited thereto, and the heat insulation reinforcement portion may be provided in the first to third surfaces 1211 to 1213, as in the first modification illustrated in FIG. 7.

Even in a case in which the configuration of the first modification described above is adopted, the same effects as those of the above-described embodiment are obtained.

### Second Modification

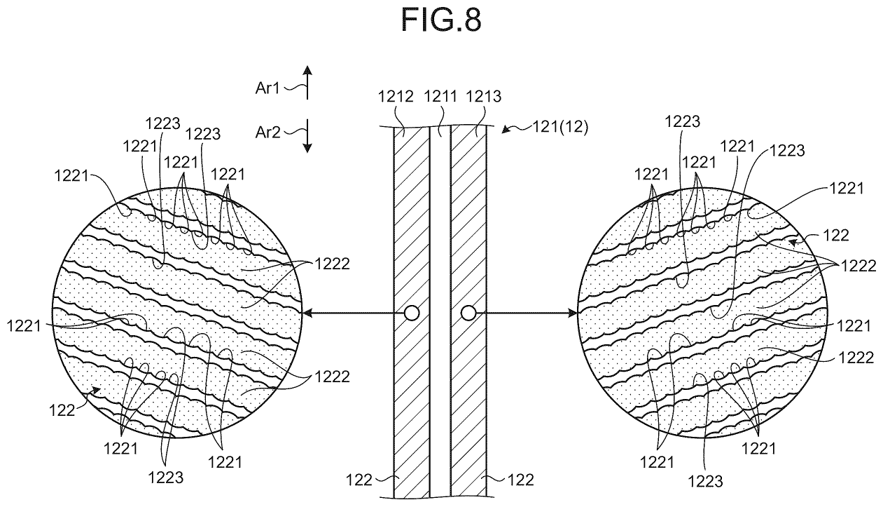

FIG. 8 is a diagram illustrating a second modification of the embodiment. Specifically, FIG. 8 is a diagram corresponding to FIG. 4. It is noted that, in FIG. 8, a portion in which the heat insulation reinforcement portion 122 is provided is hatched. In addition, in FIG. 8, a dot is added to a portion in which the first resin 1222 is disposed.

In the above-described embodiment, the plurality of groove portions 1223 constituting the heat insulation reinforcement portion 122 extend in a direction orthogonal to the longitudinal direction of the vibration transmission portion 12 and are arranged in parallel with each other in the longitudinal direction. However, the disclosure is not limited thereto.

The plurality of groove portions 1223 may extend in directions forming an angle other than 90° with respect to the longitudinal direction of the vibration transmission portion 12 and may be arranged in parallel with each other in the longitudinal direction, for example, as in the present second modification illustrated in FIG. 8.

Even in a case in which the configuration of the second modification described above is adopted, the same effects as those of the above-described embodiment are obtained.

### Third Modification

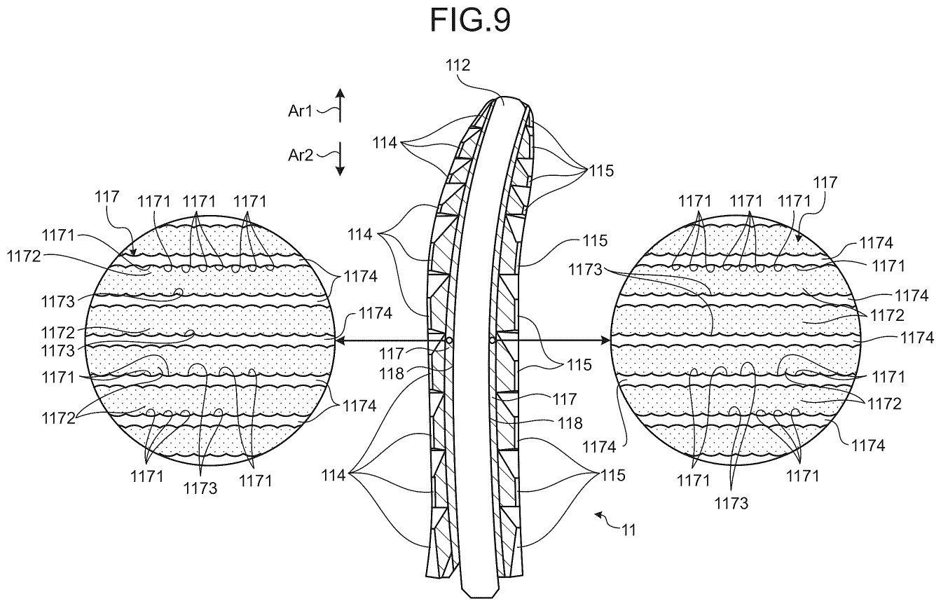

FIG. 9 is a diagram illustrating a third modification of the embodiment. Specifically, FIG. 9 is a diagram of the jaw 11 viewed from the closed side Ar4. It is noted that, in FIG. 9, a portion in which a heat insulation reinforcement portion 117 is provided is hatched.

In the above-described embodiment, as in the third modification illustrated in FIG. 9, the heat insulation reinforcement portion 117 may be provided for the jaw 11.

As illustrated in FIG. 9, in a surface of the jaw body 111 on the closed side Ar4, the heat insulation reinforcement portions 117 are provided in a pair of regions 118 of the plurality of first tooth portions 114 and the plurality of second tooth portions 115 located on both sides of the pad 112 in the width direction, the pair of regions 118 facing the treatment surfaces 1210. It is noted that the surface of the jaw body 111 on the closed side Ar4 corresponds to the treatment surface.

Similarly to the heat insulation reinforcement portion 122, the heat insulation reinforcement portion 117 is a portion that enhances a heat insulation effect of the pair of regions 118 in the jaw 11. As illustrated in FIG. 9, the heat insulation reinforcement portion 117 includes a plurality of recessed portions 1171 and first resins 1172 respectively disposed inside the plurality of recessed portions 1171.

In the third modification, the plurality of recessed portions 1171 are respectively formed in the pair of regions 118 by laser processing using a short pulse laser, and each of the recessed portions has a circular shape. In the example of FIG. 9, a plurality of rows (hereinafter, referred to as a groove portion 1173) of the plurality of recessed portions 1171 arranged in a state in which the plurality of recessed portions 1171 overlap each other in a direction orthogonal to the longitudinal direction of the jaw 11 (in a state of communicating with each other) are arranged in parallel with each other in the longitudinal direction.

It is noted that, in the following description, in the pair of regions 118, a portion other than a portion in which the groove portion 1173 is formed is referred to as a surface 1174 (FIG. 9).

Each of the first resins 1172 is a material having electrical insulation properties, and is made of a fluororesin such as PEEK, PI, PFA, or PTFE. The first resins 1172 are respectively disposed inside the groove portion 1173 in a state of being substantially flush with the surface 1174.

Then, in the third modification, the surface 1174 of the pair of regions 118, which is not covered with the first resins 1172, does not limit the path of the high-frequency current to the treatment target by the first resins 1172, and functions as an electrode (the first electrode or the second electrode) that treats the treatment target by supplying the high-frequency current to the treatment target. In addition, when the surface 1174 functions as the first electrode, the vibration transmission portion can function as the second electrode that supplies the high-frequency current to the treatment target between the first electrode and the second electrode.

It is noted that a method of forming the heat insulation reinforcement portion 117 is the same as the method of forming the heat insulation reinforcement portion 122, and thus the description thereof is omitted.

Even in a case in which the configuration of the third modification described above is adopted, the same effects as those of the above-described embodiment are obtained.

It is noted that the treatment tool 2 may be configured to have both the heat insulation reinforcement portions 117 and 122, or may be configured to have only the heat insulation reinforcement portion 117 out of the heat insulation reinforcement portions 117 and 122.

### Fourth Modification

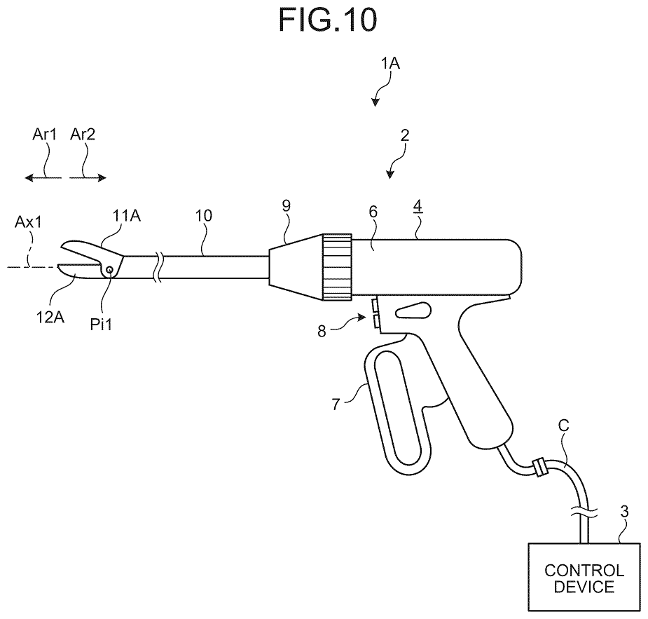

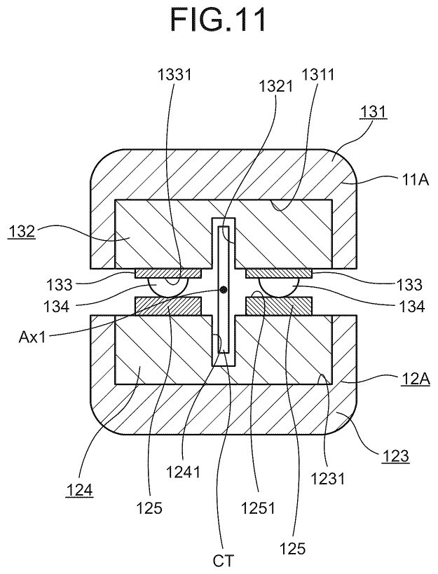

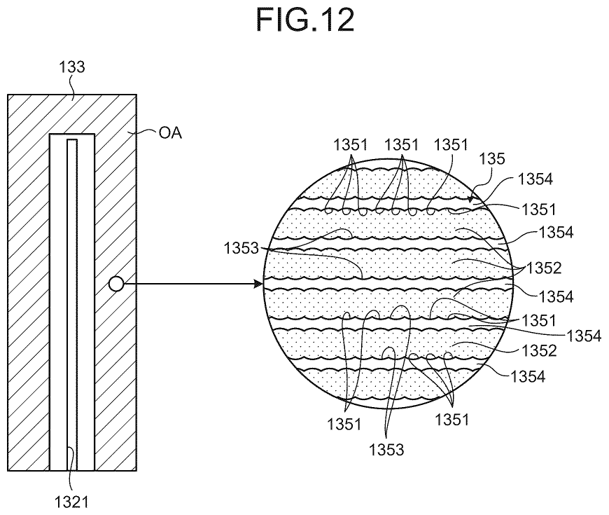

FIGS. 10 to 12 are diagrams for explaining a fourth modification of the embodiment. Specifically, FIG. 10 is a diagram corresponding to FIG. 1, and is a diagram illustrating a treatment system 1A according to the fourth modification. FIG. 11 is a diagram corresponding to FIG. 3, and is a cross-sectional view of first and second jaws 11A and 12A taken along a plane orthogonal to the central axis Ax1. FIG. 12 is a diagram of a first treatment portion 133 as viewed from the second jaw 12A side. It is noted that, in FIG. 12, a region OA is hatched.

In the treatment system 1A according to the fourth modification, as illustrated in FIG. 10 or 11, the first jaw 11A is adopted instead of the jaw 11, and the second jaw 12A is adopted instead of the vibration transmission portion 12. The first and second jaws 11A and 12A correspond to a pair of jaws.

As illustrated in FIG. 11, the first jaw 11A includes a first jaw body 131, a first support 132, a first treatment portion 133, and an abutting portion 134.

The first jaw body 131 is formed in an elongated shape extending along the central axis Ax1. The end portion of the proximal end side Ar2 of the first jaw body 131 is pivotally supported with respect to the sheath 10 by the first pin Pi1. Then, the first jaw 11A rotates about the central axis of the first pin Pi1 in conjunction with movement of the opening and closing mechanism D1 to the distal end side Ar1 or the proximal end side Ar2 according to the opening and closing operations to the operation handle 7 by an operator such as a technician, and is opened and closed with respect to the second jaw 12A. In order to have predetermined rigidity, a part of the first jaw body 131 is made of a metal material such as a titanium alloy.

In the first jaw body 131, as illustrated in FIG. 11, a storage recessed portion 1311 located at the center in the width direction and formed to extend along the central axis Ax1 is provided on the surface on the second jaw 12A side.

The first support 132 is an elongated flat plate extending along the central axis Ax1, and has substantially the same outer shape as the inner shape of the storage recessed portion 1311. The first support 132 is fitted into the storage recessed portion 1311. The first support 132 is made of, for example, an electrically insulating material having a low thermal conductivity, such as PEEK. The first support 132 is then disposed between the first treatment portion 133 and the first jaw body 131. That is, by providing the first support 132, the first jaw body 131 and the first treatment portion 133 are electrically insulated.

In the first support 132, as illustrated in FIG. 11, a cutter groove portion 1321 extending along the central axis Ax1 is provided in a substantially central portion of the surface located on the second jaw 12A side in the width direction.

The first treatment portion 133 is made of an electrically conductive material, and is a portion to which high-frequency power is supplied from a power supply (not illustrated) to a second treatment portion 125 (FIG. 11) constituting the second jaw 12A under the control of the control device 3. The first treatment portion 133 is a flat plate having a U shape that planarly surrounds the cutter groove portion 1321. Then, the first treatment portion 133 is fixed to the surface of the first support 132 on the second jaw 12A side in a posture in which both ends of the U-shape face the proximal end side Ar2.

The surface of the first treatment portion 133 on the second jaw 12A side corresponds to a treatment surface 1331. As illustrated in FIG. 12, a heat insulation reinforcement portion 135 is provided in a part of the treatment surface 1331. In the fourth modification, the heat insulation reinforcement portion 135 is provided in a U-shaped outer region OA (FIG. 12) of the surface of the first treatment portion 133 on the second jaw 12A side. It is noted that a position at which the heat insulation reinforcement portion 135 is provided may be a region other than the region OA described above.

Similarly to the heat insulation reinforcement portion 122, the heat insulation reinforcement portion 135 is a portion that enhances a heat insulation effect of the treatment surface 1331. As illustrated in FIG. 12, the heat insulation reinforcement portion 135 includes a plurality of recessed portions 1351 and first resins 1352 respectively disposed inside the plurality of recessed portions 1351.

In the fourth modification, the plurality of recessed portions 1351 are respectively formed in the region OA by laser processing using a short pulse laser, and each of the recessed portions has a circular shape. In the example of FIG. 12, a plurality of rows (hereinafter, referred to as groove portions 1353) of the plurality of recessed portions 1351 arranged in a state in which the plurality of recessed portions 1351 overlap each other (in a state of communicating with each other) in a direction orthogonal to the longitudinal direction of the first jaw 11A (extending direction of the cutter groove portion 1321) are arranged in parallel with each other in the longitudinal direction.

It is noted that, in the following description, in the treatment surface 1331, a portion other than a portion in which the groove portion 1353 is formed is referred to as a surface 1354 (FIG. 12).

Each of the first resins 1352 is a material having electrical insulation properties and is made of a fluororesin such as PEEK, PI, PFA, or PTFE. The first resins 1352 are respectively disposed inside the groove portions 1353 in a state of being substantially flush with the surface 1354.

The abutting portion 134 has a hemispherical shape made of an electrically insulating material, and is provided on a surface of the first treatment portion 133 on the second jaw 12A side. Then, the abutting portion 134 abuts on the second treatment portion 125 when the first jaw 11A is closed with respect to the second jaw 12A. That is, the abutting portion 134 prevents a short circuit between the first and second treatment portions 133 and 125.

As illustrated in FIG. 11, the second jaw 12A includes a second jaw body 123, a second support 124, and a second treatment portion 125.

The second jaw body 123 is a portion extending a part of the sheath 10 toward the distal end side Ar1, and is formed in an elongated shape extending along the central axis Ax1.

In the second jaw body 123, as illustrated in FIG. 11, a storage recessed portion 1231 located at the center in the width direction and formed to extend along the central axis Ax1 is provided on the surface on the first jaw 11A side.

The second support 124 is an elongated flat plate extending along the central axis Ax1, and has substantially the same outer shape as the inner shape of the storage recessed portion 1231. The second support 124 is fitted into the storage recessed portion 1231. The second support 124 is made of, for example, an electrically insulating material having a low thermal conductivity, such as PEEK. The second support 124 is then disposed between the second treatment portion 125 and the second jaw body 123. That is, by providing the second support 124, the second jaw body 123 and the second treatment portion 125 are electrically insulated.

In the second support 124, as illustrated in FIG. 11, a cutter groove portion 1241 extending along the central axis Ax1 and facing the cutter groove portion 1321 when the first jaw 11A is closed with respect to the second jaw 12A is provided in a substantially central portion of the surface on the first jaw 11A side in the width direction.

The second treatment portion 125 is made of an electrically conductive material, and is a portion to which high-frequency power is supplied from a power supply (not illustrated) to the first treatment portion 133 under the control of the control device 3. The second treatment portion 125 is a flat plate having a U shape that planarly surrounds the cutter groove portion 1241. Then, the second treatment portion 125 is fixed to the surface of the second support 124 on the first jaw 11A side in a posture in which both ends of the U-shape face the proximal end side Ar2.

It is noted that, hereinafter, a surface of the second treatment portion 125 on the first jaw 11A side is referred to as a treatment surface 1251.

Then, when applying high-frequency energy to the treatment target, the control device 3 supplies high-frequency power from a power supply (not illustrated) to the first and second treatment portions 133 and 125 via the electric cable C. As a result, a high-frequency current is supplied to the treatment target gripped between the treatment surfaces 1331 and 1251 from the surface (including the surface 1354) of the treatment surface 1331 that is not covered by the first resins 1352 and the treatment surface 1251. In other words, the high-frequency energy is applied to the treatment target. Therefore, the surface (including the surface 1354) of the treatment surface 1331, which is not covered with the first resins 1352, functions as the first electrode without limiting the path of the high-frequency current to the treatment target by the first resins 1352.

In addition, in the fourth modification, as illustrated in FIG. 11, the treatment tool 2 is provided with a cutter CT which is positioned in the cutter groove portions 1321 and 1241 and moves forwards and rearwards along the central axis Ax1 according to the operation of an operation lever (not illustrated) by an operator such as a technician. That is, the treatment target gripped between the first and second jaws 11A and 12A is incised by the forward-and-rearward movement of the cutter CT.

Even in a case in which the configuration of the fourth modification described above is adopted, the same effects as those of the above-described embodiment are obtained.

It is noted that, in the fourth modification described above, the heat insulation reinforcement portion 135 is provided only on the treatment surface 1331, but the disclosure is not limited thereto, and the heat insulation reinforcement portion may be provided on both of the treatment surfaces 1331 and 1251 or only on the treatment surface 1251.

According to a treatment tool and a manufacturing method according to the disclosure, it is possible to supply a high-frequency current to a treatment target while reducing residual heat on the treatment surface.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A treatment tool comprising:
a treatment surface for treating of a treatment target; and
a plurality of recessed portions that are provided in at least a part of the treatment surface and in which first resins are disposed, the treatment surface including a surface region not covered with the first resins, the surface region functioning as a first electrode supplying a high-frequency current to the treatment target to treat the treatment target.

2. The treatment tool according to claim 1, further comprising a vibration transmission portion configured to treat the treatment target with ultrasonic vibration,
wherein the treatment surface is provided in the vibration transmission portion.

3. The treatment tool according to claim 2, further comprising a jaw configured to grip the treatment target between the treatment surface and the jaw, the jaw including a second electrode supplying the high-frequency current to the treatment target between the first electrode and the second electrode.

4. The treatment tool according to claim 3, wherein the treatment surface is a surface facing the jaw.

5. The treatment tool according to claim 4, wherein the vibration transmission portion includes a surface that is located on an opposite side of the treatment surface and is coated with a second resin.

6. The treatment tool according to claim 5, wherein at least a part of an outer surface of the vibration transmission portion, excluding the treatment surface, is coated with the second resin, the part of the outer surface being located on a distal end side from a most distal node position.

7. The treatment tool according to claim 3, wherein
the treatment surface includes an abutting surface abutting on the jaw when the jaw is closed with respect to the treatment surface, and
the abutting surface does not include the plurality of recessed portions.

8. The treatment tool according to claim 2, wherein the vibration transmission portion is made of a titanium alloy.

9. The treatment tool according to claim 1, wherein each of the first resins is made of polyether ether ketone, polytetrafluoroethylene, perfluoroalkoxy alkane, or polyimide.

10. The treatment tool according to claim 1, further comprising:
a vibration transmission portion configured to treat the treatment target with ultrasonic vibration; and
a jaw configured to grip the treatment target between the vibration transmission portion and the jaw,
wherein the treatment surface is provided in the jaw, and
the vibration transmission portion functions as a second electrode supplying the high-frequency current to the treatment target between the first electrode and the second electrode.

11. The treatment tool according to claim 1, further comprising a pair of jaws configured to grip the treatment target,
wherein the treatment surface is provided in at least one of the pair of jaws.

12. A method of manufacturing a treatment tool including a treatment surface for treatment of a treatment target, the method comprising:
providing a plurality of recessed portions in the treatment surface;
applying first resins to the treatment surface; and
removing, among the first resins applied to the treatment surface, resins other than the first resins disposed in the plurality of recessed portions.

13. The method according to claim 12, wherein the plurality of recessed portions are formed by a laser.
